(19) United States
(12) Patent Application Publication
(10) Pub. No.: US 2005/0148081 A1
(43) Pub. Date: Jul. 7, 2005

(54) System and method for corrosion maintenance scheduling

(76) Inventors: Russell D. Braunling, Eden Prairie, MN (US); Paul F. Dietrich, Brooklyn Park, MN (US); Darryl J. Wrest, Coon Rapids, MN (US)

Correspondence Address: Matthew S. Luxton;Honeywell International, Inc., Law Dept. AB2, 101 Columbia Road, Morristown, NJ, 07962, US

(21) Appl. No.: 10/751,449

(22) Filed: Jan. 6, 2004

(51) Int. Cl. G01N 33/26

(52) U.S. Cl. 436/6; 422/53

(57) ABSTRACT

A system for scheduling maintenance on equipment includes a metallic element placed on or near a piece of equipment in an environment, a measuring and data storing device configured to measure the resistance of the metallic element, and a computer configured to determine the amount of corrosion experienced by the metallic element based on the resistance measured by the measuring and data storing device and to correlate the amount of corrosion with a maintenance schedule for the piece of equipment. A method for scheduling maintenance on a piece of equipment is also described. The method includes placing a metallic element on or near the piece of equipment, determining the amount of corrosion experienced by the metallic element, and correlating the amount of corrosion with a maintenance schedule for the piece of equipment.

This invention was made with Government support under contract no. N00014-02-C-0147 awarded by the Office of Naval Research. The Government has certain rights in the invention.

### BACKGROUND

1. Field of the Invention

The present invention generally relates to systems and methods for monitoring an environment and scheduling maintenance based on that monitoring. More particularly, the invention can be used to monitor the corrosive environment for a particular device, machine, or structure and schedule maintenance based on the monitored environment.

2. Background of the Invention

Corrosion can lead to failures in infrastructure, machines, and mission critical systems. Such failures are expensive to repair, can lead to soiled products, contaminated products, or products beyond salvage. The failures can cause environmental damage, and ultimately, can even cause unsafe environments or situations for humans. Decisions regarding the future integrity of a structure or its components depend substantially upon an accurate assessment of the conditions affecting its corrosion and rate of deterioration. Only with accurate information in hand, can an owner or operator make an informed decision as to the type, cost, and urgency of repair or replacement.

Corrosion monitoring is particularly important in areas that cannot be readily inspected visually or are difficult to inspect due to the inherent structural arrangement of a particular device, machine or structure. For example, there may be cavities within vehicles that are generally not accessible because of equipment or other structures that block an opening to the cavity. Nevertheless, corrosion monitoring of such spaces is desirable, and perhaps critical.

One well-known method of monitoring corrosion is the Electrical Resistance technique. This technique effectively measures material loss, i.e., corrosion, by measuring a change in electrical resistance of a metallic element, which is exposed to a selected environment, with respect to a reference element that is arranged to be immune from that environment's corrosive effects. While this technique is very popular and has found wide acceptance, the technique by itself does not provide guidance as when to perform (i.e., schedule) maintenance of the particular device, machine, or structure.

### BRIEF SUMMARY OF THE INVENTION

The present invention uses a metallic element, such as a thin metal strip (or corrosion coupon), to continuously measure the effects of the corrosive environment. The metal used in the coupon is chosen to have high environmental sensitivity to corrosion and is typically different from the metal in the asset that it is monitoring. Corrosion data is obtained from the coupon using the Electrical Resistance technique to provide corrosion rate information. The invention uses the corrosion rate information to calculate the change in thickness of the metal in mils over time. The calculated thickness loss is compared to the expected thickness loss for the assumed environment used in a calendar based maintenance schedule. A Corrosivity Ratio is calculated that compares the actual loss in thickness with the expected loss in thickness of the metal strip. A look-up table may be used to provide the recommended delay in maintenance scheduling based on the Corrosivity Ratio. This results in condition-based maintenance of the particular device, machine, or structure. The invention preferably also uses temperature and humidity data to remove unsatisfactory Electrical Resistance estimates for the corrosion rate from being used in the calculation of the Corrosivity Ratio.

An exemplary embodiment of the invention uses a carbon steel coupon. Carbon steel is selected because it is very sensitive to the corrosive environment and estimates of thickness (or weight) loss are minimally affected by corrosion product buildup on the surface of the coupon. Other metals and alloys could also be used for the coupon as long as they are more sensitive to the environment then the metal in the asset and are minimally affected by corrosion products.

The features and attendant advantages of the present invention will be more fully appreciated upon a reading of the following detailed description in conjunction with the accompanying drawings.

### BRIEF DESCRIPTION OF THE DRAWINGS

FIG. 1 is a schematic drawing of an exemplary system in accordance with the present invention.

FIG. 2 shows an exemplary method in accordance with the present invention that can be performed using the system of FIG. 1.

FIGS. 3A and 3B show exemplary look-up tables accessed in connection with performing the method according to FIG. 2.

### DETAILED DESCRIPTION OF THE INVENTION

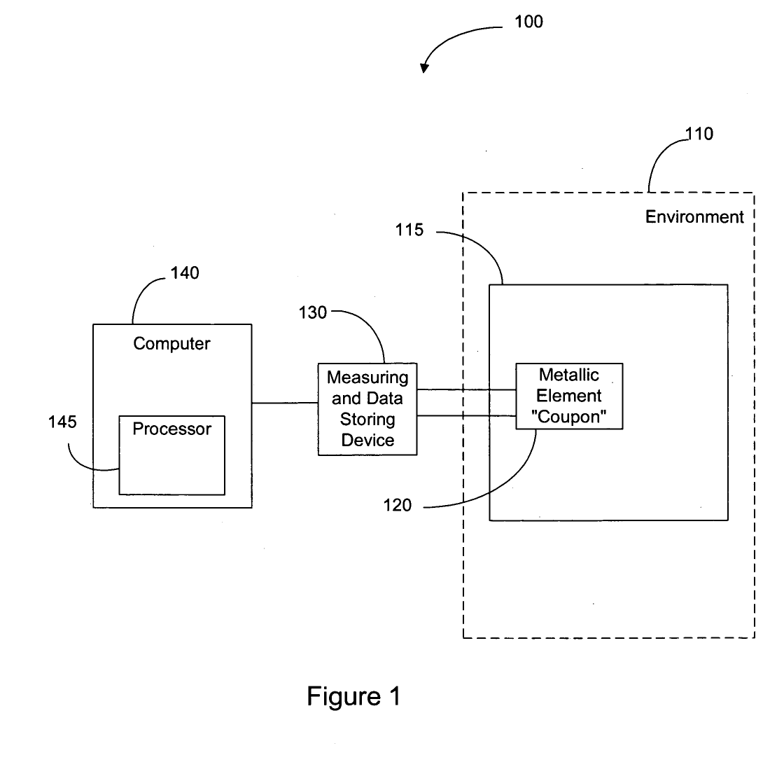

FIG. 1 shows an exemplary system 100 of the present invention. System 100 includes an environment 110, which may be any environment where it is desirable to determine the corrosive effects on a piece of equipment 115, such as a particular device, machine, or structure located therein. A metallic element 120, which may take the form of a thin metallic strip, is placed in environment 110 or on a particular device, machine, or structure located in environment 110.

Metallic element 120 may be a test coupon as described in co-pending patent application Ser. No. 10/383,689, which is hereby incorporated by reference in its entirety. The metallic element is configured to have a test portion exposed to the environment and reference portion that is sealed from the conditions of the environment. Therefore, only the test portion is exposed to the elements and experiences corrosion. The metallic element may be made of carbon steel. Carbon steel is sensitive to the corrosive environment and estimates of thickness (or weight) loss are minimally affected by corrosion product buildup on the surface of the carbon steel. Other metals and alloys could also be used for metallic element 120 as long as they are more sensitive to the environment then the metal in the particular device, machine, or structure and are minimally affected by corrosion products.

Metallic element 120 is connected to a measuring and data storing device 130, also described in co-pending patent application Ser. No. 10/383,689, which is configured to measure the resistance of the test portion along with the resistance of the reference portion. The measured resistances are downloaded to a computer 140.

Computer 140 may be any type of computer, but a handheld computer may be the most appropriate depending on the location of measuring and data storing device 130 and the particular environment 110. Computer 140 includes a processor 145 that is configured to receive the resistance measurements of metallic element 120 and convert the resistances into a corrosion rate for metallic element 120. The corrosion rate is then used to calculate the amount of corrosion of metallic element 120 and is further used to correlate the amount of corrosion of metallic element 120 with a maintenance schedule. There are numerous approaches to correlating the corrosion loss to a maintenance schedule.

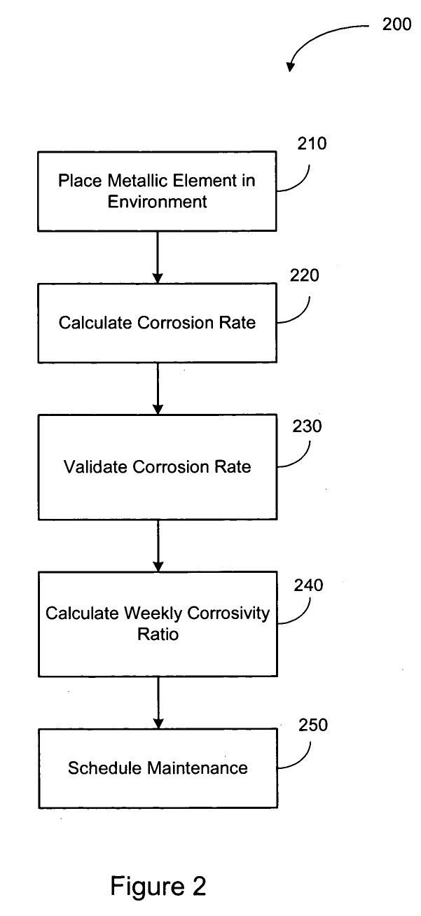

FIG. 2 shows an exemplary method 200 that is preferably employed in connection with system 100 described above. First, at step 210, metallic element 120 is placed in environment 110 or on a particular device, machine, or structure located in environment 110.

At step 220, the corrosion rate is calculated periodically by processor 145. For example, the corrosion rate may be calculated daily. Alternatively, the period could vary depending on the type of equipment that is monitored (i.e., delicate equipment may require more continuous monitoring). To calculate the corrosion rate, the measuring and data storing device measures both the resistance of the test portion (Rₜₑₛₜ) and the reference portion (R_{reference}) of the metallic element 120. The processor 145 determines and stores the ratio of Rₜₑₛₜ and R_{reference}.

Specifically, the processor calculates the corrosion rate using the following equation:
C_{R}=[(S₂−S₁)×P/K]/(ΔT/365);
where C_{R} is measured in “mils lost” per year; S₂ and S₁ are ratios of Rₜₑₛₜ/R_{reference} measured at times T₂ and T₁, respectively; P is the span of the metallic element 120 measured in mils; K is a constant that depends on the electronic configuration; and ΔT is the difference of T₂−T₁ in days.

Before the actual C_{R} can be calculated, it is necessary to solve for K based on the particular configuration of the metallic element and associated circuitry. For example, where metallic element 120 is formed of carbon steel having a thickness of 8 mils, which is twice span P, and it takes ½ a year for the span to be consumed, the C_{R} is equal to 8 mils lost/year. Solving for K, assuming no resistance measurement error (i.e., S₁=1 at T₁ and S₂, which is inversely proportional to the metal thickness, =2 at T₂) and S₂=2 when the entire span is consumed, then:
8=365×4(2−1)/182.5K, which implies K=1.
   As a result, the processor can calculate C_{R} using the following equation:
   C_{R}=1460(S₂−S₁)/ΔT.

As another example, if the resistance scale was calibrated to output on a 0 to 1000 scale over the life of the sensor, then S₂−S₁=1000, K=1000 and the processor would calculate C_{R} using the following equation:
C_{R}=0.365P(S₂−S₁)/(ΔT).

Once the processor 145 is programmed with the proper equation for C_{R}, it is possible to create, e.g., a weekly corrosion rate estimate by summing the measured C_{R}_{^{S}} and finding the average as shown in the following equation:

$${W}_{n} = {\sum }_{i = 7 ( n - 1 ) + 1}^{7 n} {C}_{Ri} / 7.$$

For the above equation, Wₙ is the weekly average of each day's corrosion rate estimate for the n^{th} week and C_{Ri} is the C_{R} measured for a particular day during the week.

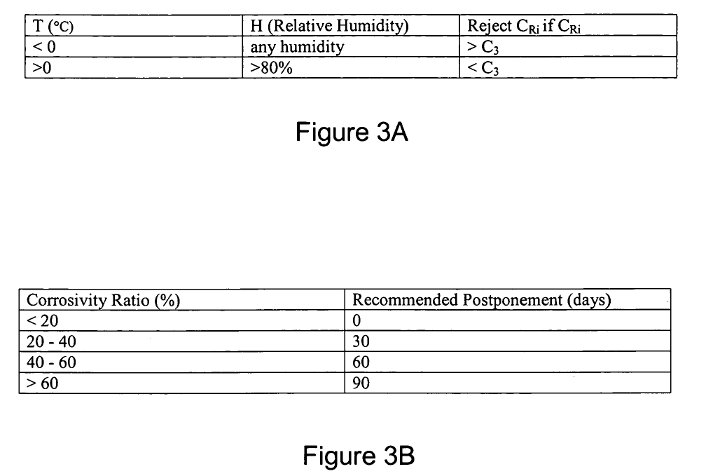

Depending on the conditions in environment 110, an individual C_{Ri} may not be reliable. For example, temperature and humidity should correlate with the measurements obtained by measuring and data storing device 130 and metallic element 120 (i.e., high temperature and high humidity should correspond to a high corrosion rate). Therefore, at step 230, the data is validated by using a look-up table. FIG. 3A shows a simple look-up table for validating the calculated C_{Ri} based on the temperature and humidity of the environment. The calculated C_{Ri} is compared to a reference value obtained from the look-up table using the measured values for temperature and humidity. Depending on the comparison, if the C_{Ri} is greater or less than a reference value, the calculated C_{Ri} is ignored and the previous C_{Ri} is used for the calculation. For example, if the temperature was greater than 0 degrees Centigrade and the humidity was greater than 80%, then the calculated C_{Ri} would have to be greater than a reference value C₃ to be an acceptable measurement. The reference values C₃ for carbon steel is obtained form International Standard (ISO) 9223 as 25 micrometers/year or 0.98 mils/year. Although a look-up table has been shown, it is understood that many other validation techniques may be used to remove outliers from the data. It is also understood that C₃ could vary depending on the metal used.

Once the weekly corrosion rate W has been calculated, it is possible, at step 240, to calculate the cumulative numbers of mils lost and correlate this with a maintenance schedule. The following equation is used to calculate the mils lost over n number of weeks:

$$ML = {\sum }_{i = 1}^{n} {W}_{i} * 7 / 365.$$

Once ML is calculated, there are a number of different ways to use ML to determine when maintenance should be scheduled. For example, assuming that the metallic element has a “worst case” constant corrosion rate WC_{R}, in mils/year, it is possible to calculate a corrosivity ratio, in percent; using the following equation:
Corrosivity Ratio=[((WC_{R}*7/365)n−ML)/(WC_{R}*7/365)n]*100.

This corrosivity ratio is then used to determine when maintenance should be performed on a particular device, machine, or structure at step 250. FIG. 3B shows a look-up table that correlates the corrosivity ratio with the number of days that maintenance should be delayed. For example, if the corrosivity ratio is less than 20%, then maintenance should be scheduled immediately. Conversely, if the corrosivity ratio is greater than 60%, then maintenance can be postponed 90 days. These time delays may vary depending on the particular device, machine, or structure and the correlation drawn between the corrosion rate of metallic element 120 and the particular device, machine, or structure. Alternatively, the actual mils lost may be used to correlate with the maintenance schedule without requiring a calculation of a corrosivity ratio.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

1. A method for scheduling maintenance on a piece of equipment, comprising:
placing a metallic element having a test portion and a reference portion in an environment in which the piece of equipment is located;
determining the amount of corrosion experienced by the metallic element by measuring the resistance of the test portion and the reference portion; and
correlating the amount of corrosion with a maintenance schedule for the piece of equipment.

2. The method according to claim 1, wherein determining the amount of corrosion includes periodically measuring the corrosive rate of the metallic element.

3. The method according to claim 2, wherein measuring the corrosive rate of the metallic element is performed daily.

4. The method according to claim 2, wherein the amount of corrosion is compared to an expected amount of corrosion, and a resulting comparison result of the amount of corrosion and the expected amount of corrosion is used to determine the maintenance schedule for the equipment.

5. The method according to claim 1, wherein determining the amount of corrosion experienced by the metallic element is validated based on conditions of the environment.

6. The method according to claim 5, wherein the conditions of the environment comprise the temperature of the environment.

7. The method according to claim 5, wherein the conditions of the environment comprise the humidity of the environment.

8. A system for scheduling maintenance on equipment, comprising:
a metallic element having a test portion and a reference portion, the metallic element to be placed in an environment in which a piece of equipment is located;
a measuring and data storing device configured to measure the resistance of the test portion and the reference portion; and
a computer configured to determine the amount of corrosion experienced by the metallic element based on the resistances measured by the measuring and data storing device and to correlate the amount of corrosion with a maintenance schedule for the piece of equipment.

9. (canceled)

10. The system according to claim 8, wherein the computer calculates a corrosion rate periodically based on the resistance of the test portion and the resistance of the reference portion.

11. The system according to claim 10, wherein the computer calculates the corrosion rate daily.

12. The system according to claim 8, wherein the computer compares the amount of corrosion to an expected amount of corrosion, and a comparison result of the amount of corrosion and the expected amount of corrosion is used to determine a delay of maintenance for the piece of equipment.

13. The system according to claim 12, wherein the computer uses the comparison result of the amount of corrosion and the expected amount of corrosion to perform a table look-up to determine the delay of maintenance for the equipment.

14. The system according to claim 8, wherein the computer validates the amount of corrosion experienced by the metallic element based on conditions of the environment.

15. The system according to claim 14, wherein the conditions of the environment comprise the temperature of the environment.

16. The system according to claim 15, wherein the conditions of the environment comprise the humidity of the environment.

17. The system according to claim 8, wherein the metallic element is formed from carbon steel.

18. The system according to claim 2, wherein the corrosion rate is calculated based on the resistance of the test portion and the reference portion.
